(19) United States
(12) Patent Application Publication
(10) Pub. No.: US 2025/0366824 A1
(43) Pub. Date: Dec. 4, 2025

(54) ULTRASOUND PROBE

(71) Applicant: FUJIFILM Corporation, Tokyo (JP)

(72) Inventor: Hiroki TANAKA, Tokyo (JP)

(73) Assignee: FUJIFILM Corporation, Tokyo (JP)

(21) Appl. No.: 19/208,501

(22) Filed: May 14, 2025

(30) Foreign Application Priority Data
May 29, 2024 (JP) 2024-087283

(51) Int. Cl. A61B 8/00

(52) U.S. Cl. CPC A61B 8/4444; A61B 8/4494

(57) ABSTRACT

The ultrasound probe has a vibration element array 10 including a plurality of vibration elements 12 arranged in an array direction. The vibration element 12 is virtually divided into a first virtual portion 12a and a second virtual portion 12b by a straight line 22 that passes through the centroid 20 of the vibration element 12 and extends in the array direction. The vibration element 12 has a shape in which a first virtual centroid 24a that is a centroid of the first virtual portion 12a and a second virtual centroid 24b that is a centroid of the second virtual portion 12b are at different positions in the array direction.

### CROSS REFERENCE TO RELATED APPLICATION

This application claims priority to Japanese Patent Application No. 2024-087283 filed on May 29, 2024, which is incorporated herein by reference in their entireties including the specifications, claims, drawings, and abstracts.

### BACKGROUND OF THE INVENTION

### 1. Field of the Invention

The present specification discloses improvement of an ultrasound probe.

### 2. Description of the Related Art

An ultrasound diagnostic apparatus that can form an ultrasound tomographic image representing a cross section in an ultrasound scanning plane and perform various measurements based on a reflected waves from a subject in a case where an ultrasound waves are transmitted to the subject is known. The ultrasound diagnostic apparatus comprises an ultrasound probe that is in contact with a subject and transmits and receives an ultrasound waves to and from the subject. The ultrasound probe is provided with a plurality of vibration elements, and the transmission signals are supplied from an apparatus main body of the ultrasound diagnostic apparatus to each of the plurality of vibration elements, so that ultrasound waves are transmitted from the plurality of vibration elements toward the subject. In addition, the plurality of vibration elements receive reflected waves from the subject, and transmit a reception signal obtained by converting the reflected waves into an electric signal to the apparatus main body.

In the related art, various improvement proposals for the ultrasound probe, in particular, improvement proposals for the arrangement of a plurality of vibration elements have been proposed. For example, JP1998-62396A (JP-H10-62396A) discloses an ultrasound probe in which vibration elements are disposed in an inclined manner in an array direction in order to obtain a two-dimensional image or a three-dimensional image in real time by an ultrasound probe in which the vibration elements are arranged in a one-dimensional direction (array direction). In addition, JP1999-64492A (JP-H11-64492A) discloses a cylindrical tubular ultrasound transducer in which an ultrasound transducer is provided to be inclined with respect to a circumferential direction.

### SUMMARY OF THE INVENTION

By the way, a virtual image may be displayed in an ultrasound tomographic image formed by an ultrasound diagnostic apparatus. Such a virtual image is also referred to as an artifact. One of the causes of the artifact is a grating beam. The grating beam will be described with reference to FIGS. 45 to 48.

FIG. 45 is a plan view of a vibration element array PEA provided in an ultrasound probe in the related art, and FIG. 46 is a side view of the vibration element array PEA in the related art. In the present specification, a plan view means a view as seen from a depth direction, and a side view means a view as seen from an elevation direction. In addition, in the present specification, a direction in which a plurality of vibration elements PE are arranged is referred to as an array direction, a direction that is parallel (same plane) to the vibration element surface and is perpendicular to the array direction is referred to as an elevation direction, and a direction perpendicular to the array direction and the elevation direction is referred to as a depth direction.

As shown in FIG. 45, here, it is assumed that the vibration element PE in the related art has a substantially rectangular shape extending in the elevation direction. In a case where the transmission signals are supplied to each vibration element PE, an ultrasound waves are output from each vibration element PE. In FIG. 46, the wavefront WF of the ultrasound waves output from each vibration element PE is represented by an arc centered on each vibration element PE. The wavefronts WF from each vibration element PE are combined to form a composite wavefront CWFm, and the ultrasound waves have a property of a beam. A direction indicated by an arrow Dm perpendicular to the composite wavefront CWFm is the transmission direction of the ultrasound beam. The ultrasound beam intended to be output (target) is called a main beam. As shown in FIG. 46, in a case where ultrasound waves are simultaneously output from each vibration element PE, the direction of the main beam is a direction parallel to the depth direction. However, the direction of the main beam can be controlled by controlling the output timing of the ultrasound waves from each vibration element PE.

Here, an ultrasound beam (referred to as a grating beam) may be formed in a direction different from the main beam. For example, as shown in FIG. 46, a case where ultrasound waves are simultaneously output from each vibration element PE and the direction of the main beam is a direction parallel to the depth direction is considered. A grating beam is formed by combining a wavefront WF output from a certain vibration element PE and a wavefront WF output from a vibration element PE adjacent to the certain vibration element PE with respect to the wavefront WF with a delay of one period. Each vibration element PE repeatedly outputs ultrasound waves based on a transmission frequency.

FIG. 47 is a diagram showing a principle of generating a grating beam. First, the wavefront WFa is output from the vibration element PEa. In this case, wavefronts are also output from other vibration elements PE including the vibration elements PEb and PEc, but the wavefronts are not shown in FIG. 47. After one period (1/f [s] after in a case where the transmission frequency is f), the wavefront WFb is output from the vibration element PEb. In this case, the wavefront output from the vibration element PE other than the vibration element PEb is also omitted in the drawing. Further, after one period, the wavefront WFc is output from the vibration element PEc. In this case, the wavefront output from the vibration element PE other than the vibration element PEc is also not shown.

Then, the wavefronts WFa, WFb, and WFc are combined to form a composite wavefront CWFg, which also has properties of a beam. This is a grating beam, and a transmission direction of the grating beam is a direction indicated by an arrow Dg perpendicular to the composite wavefront CWFg.

FIG. 48 is a diagram for explaining a generation condition of a grating beam. In FIG. 48, a pitch between the vibration elements PE in the vibration element array PEA (referred to as a vibration element pitch in the present specification) is indicated by P, and an angle of the direction of the main beam (the direction of the arrow Dm) with respect to the depth direction is indicated by θm. In a case where the plurality of vibration elements PE are arranged on a plane, a generation condition of a grating beam is represented by the following Expression 1.

$$P > λ / ( 1 + sin θ m ) \qquad Expression 1$$

In Expression 1, λ is a wavelength of the ultrasound wave output from each vibration element PE and is the reciprocal of the transmission frequency of the ultrasound wave.

From Expression 1, it can be said that the smaller the transmission frequency of the ultrasound wave is (the longer the wavelength is), the more difficult it is to generate a grating beam. However, from the viewpoint of increasing the image quality of the ultrasound tomographic image, it may not be appropriate to reduce the transmission frequency of the ultrasound wave. In addition, from Expression 1, as the vibration element pitch is decreased, the grating beam can be made difficult to be generated, but there is a limit to decreasing the vibration element pitch due to cost or manufacturing problems.

An object of the ultrasound probe disclosed in the present specification is to reduce the intensity of the grating beam without reducing the vibration element pitch of the vibration element array and without reducing the transmission frequency of the ultrasound wave.

An ultrasound probe according to the present disclosure is an ultrasound probe comprising a vibration element array including a plurality of vibration elements arranged in an array direction, in which, in a case where the vibration elements are virtually divided into a first virtual portion and a second virtual portion by a straight line passing through a centroid of the vibration elements and extending in the array direction, each of the vibration elements has a shape in which a first virtual centroid that is a centroid of the first virtual portion and a second virtual centroid that is a centroid of the second virtual portion are located at different positions in the array direction, and due to the shape, a grating beam output from the first virtual portion and a grating beam output from the second virtual portion are canceled out from each other due to a phase shift, so that an intensity of the grating beam output from each of the vibration elements is reduced.

In a plan view as seen from a depth direction, the shape of the vibration element may be a polygon with 5 or more sides, or at least one side of the vibration element may be curved.

In a plan view as seen from a depth direction, a shape of the first virtual portion and a shape of the second virtual portion may be in a point-asymmetric relationship with the centroid of the vibration element as a center.

An intervirtual centroid distance, which is a distance between the first virtual centroid and the second virtual centroid in the array direction, may be less than a vibration element pitch in the vibration element array.

In a case where the plurality of vibration elements may be arranged on a plane, the intervirtual centroid distance is half of the vibration element pitch.

In a case where the plurality of vibration elements are arranged on a curved surface, the intervirtual centroid distance may be half of a virtual pitch P′ between the vibration elements.

The virtual pitch P′ may be represented by

$$P ’ = P sin θ g / sin ( θ g - β )$$

$$β = ( N / 4 ) * α ,$$

here, P may be the vibration element pitch, θg may be a beam angle of the grating beam, N may be the number of diameter vibration elements, and a may be an angular pitch between the vibration elements.

According to the ultrasound probe disclosed in the present specification, it is possible to reduce the intensity of the grating beam without reducing the vibration element pitch of the vibration element array and without reducing the transmission frequency of the ultrasound wave.

### BRIEF DESCRIPTION OF THE DRAWINGS

FIG. 1 is a first diagram for explaining a cancellation condition of a grating beam.

FIG. 2 is a second diagram for explaining cancellation conditions of a grating beam.

FIG. 3 is a side view of a vibration element array of the ultrasound probe according to the first embodiment.

FIG. 4 is a plan view of a vibration element array of the ultrasound probe according to the first embodiment.

FIG. 5 is an enlarged plan view of the vibration element.

FIG. 6 is a diagram showing a grating beam generated in a linear array in the related art at a position in an elevation direction indicated by A in FIG. 4.

FIG. 7 is a diagram showing a grating beam generated in the vibration element array according to the first embodiment at a position in an elevation direction indicated by A of FIG. 4.

FIG. 8 is a diagram showing a grating beam generated in a linear array in the related art at a position in an elevation direction indicated by B in FIG. 4.

FIG. 9 is a diagram showing a grating beam generated in the vibration element array according to the first embodiment at a position in an elevation direction indicated by B in FIG. 4.

FIG. 10 is a diagram showing a grating beam generated in a linear array in the related art at a position in an elevation direction indicated by C in FIG. 4.

FIG. 11 is a diagram showing a grating beam generated in the vibration element array according to the first embodiment at a position in an elevation direction indicated by C in FIG. 4.

FIG. 12 is a diagram showing a grating beam generated in a linear array in the related art at a position in an elevation direction indicated by D in FIG. 4.

FIG. 13 is a diagram showing a grating beam generated in the vibration element array according to the first embodiment at a position in an elevation direction indicated by D in FIG. 4.

FIG. 14 is a diagram showing a grating beam generated in a linear array in the related art at a position in an elevation direction indicated by E in FIG. 4.

FIG. 15 is a diagram showing a grating beam generated in the vibration element array according to the first embodiment at a position in an elevation direction indicated by E in FIG. 4.

FIG. 16 is a diagram showing a grating beam at a predetermined depth for a linear array in the related art in a two-dimensional space of an array direction and an elevation direction.

FIG. 17 is a diagram showing a grating beam at a predetermined depth for the vibration element array according to the first embodiment in a two-dimensional space of an array direction and an elevation direction at the predetermined depth.

FIG. 18 is a diagram showing a grating beam generated in a linear array in the related art as a whole in an elevation direction.

FIG. 19 is a diagram showing a grating beam generated in the vibration element array according to the first embodiment as a whole in the elevation direction.

FIG. 20 is a graph showing a relationship between a signal intensity and a beam angle at a predetermined depth in the first embodiment.

FIG. 21 is a graph showing a relationship between a grating level and an intervirtual centroid distance at a beam angle at which the intensity of a grating beam is a peak in the first embodiment.

FIG. 22 is a first diagram for explaining an appropriate upper limit of the intervirtual centroid distance.

FIG. 23 is a second diagram for explaining an appropriate upper limit of the intervirtual centroid distance.

FIG. 24 is a diagram showing a modification example of the vibration element.

FIG. 25 is a side view of a vibration element array of an ultrasound probe according to a second embodiment.

FIG. 26 is a diagram showing a grating beam generated in a curved surface array in the related art at a position in an elevation direction indicated by A of FIG. 4.

FIG. 27 is a diagram showing a grating beam generated in the vibration element array according to the second embodiment at a position in an elevation direction indicated by A of FIG. 4.

FIG. 28 is a diagram showing a grating beam generated in a curved surface array in the related art at a position in an elevation direction indicated by B in FIG. 4.

FIG. 29 is a diagram showing a grating beam generated in the vibration element array according to the second embodiment at a position in an elevation direction indicated by B of FIG. 4.

FIG. 30 is a diagram showing a grating beam generated in a curved surface array in the related art at a position in an elevation direction indicated by C in FIG. 4.

FIG. 31 is a diagram showing a grating beam generated in the vibration element array according to the second embodiment at a position in an elevation direction indicated by C in FIG. 4.

FIG. 32 is a diagram showing a grating beam generated in a curved surface array in the related art at a position in an elevation direction indicated by D in FIG. 4.

FIG. 33 is a diagram showing a grating beam generated in the vibration element array according to the second embodiment at a position in an elevation direction indicated by D in FIG. 4.

FIG. 34 is a diagram showing a grating beam generated in a curved surface array in the related art at a position in an elevation direction indicated by E in FIG. 4.

FIG. 35 is a diagram showing a grating beam generated in the vibration element array according to the second embodiment at a position in an elevation direction indicated by E in FIG. 4.

FIG. 36 is a diagram showing a grating beam at a predetermined depth of a curved surface array in the related art in a two-dimensional space of an array direction and an elevation direction.

FIG. 37 is a diagram showing a grating beam at a predetermined depth in a two-dimensional space of an array direction and an elevation direction at the predetermined depth for the vibration element array according to the second embodiment.

FIG. 38 is a diagram showing a grating beam generated in a curved surface array in the related art as a whole in the elevation direction.

FIG. 39 is a diagram showing a grating beam generated in the vibration element array according to the second embodiment as a whole in the elevation direction.

FIG. 40 is a graph showing a relationship between a signal intensity and a beam angle at a predetermined depth in the second embodiment.

FIG. 41 is a graph showing a relationship between a grating level and an intervirtual centroid distance at a beam angle at which the intensity of a grating beam in the second embodiment is a peak.

FIG. 42 is a first diagram for explaining a method of calculating a virtual pitch.

FIG. 43 is a second diagram for explaining a method of calculating a virtual pitch.

FIG. 44 is a graph showing a relationship between a main beam width and an intervirtual centroid distance.

FIG. 45 is a plan view of a vibration element array provided in an ultrasound probe of the related art.

FIG. 46 is a side view of a vibration element array in the related art.

FIG. 47 is a diagram showing a principle of generating a grating beam.

FIG. 48 is a diagram for explaining a generation condition of a grating beam.

### DESCRIPTION OF THE PREFERRED EMBODIMENTS

### Cancellation Condition of Grating Beam

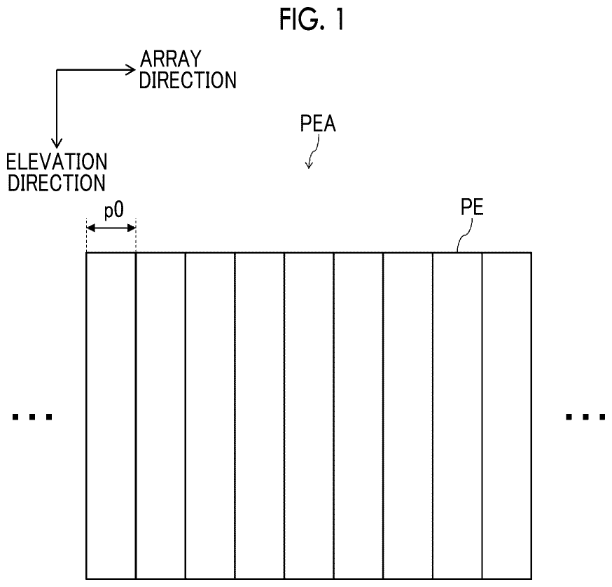

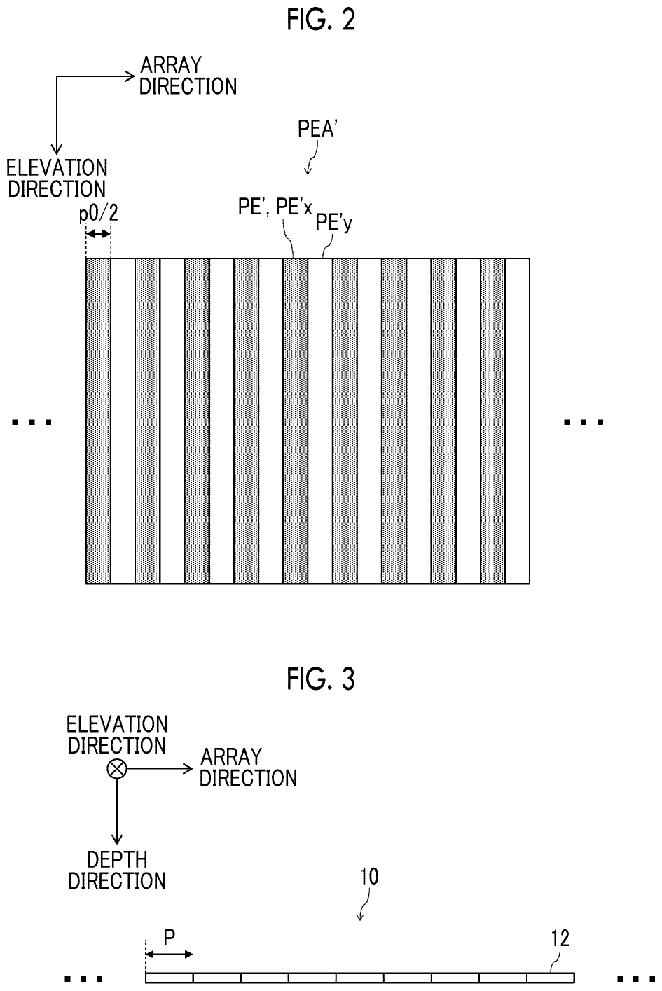

Before the description of the first embodiment, a cancellation condition of the grating beam will be described. FIGS. 1 and 2 are diagrams for explaining cancellation conditions of the grating beam. FIG. 1 is a plan view of a vibration element array PEA, which is a vibration element array PEA in which each vibration element PE has a substantially rectangular shape extending in an elevation direction.

It is assumed that the vibration element pitch of the vibration element array PEA shown in FIG. 1 is p0. Here, it is assumed that p0 satisfies the above-described generation condition of the grating beam in combination with the wavelength 2 of the ultrasound wave output from each of the vibration elements PE. That is, in a case where ultrasound waves are output from each of the vibration elements PE included in the vibration element array PEA, a grating beam is generated.

Here, a case where the vibration element pitch is set to p0/2, that is, half of the vibration element pitch satisfying the generation condition of grating beam as in the vibration element array PEA′ shown in FIG. 2 is considered. In the vibration element array PEA′ as well, a plurality of vibration elements PE are arranged in the array direction as in FIG. 1, but in the vibration element array PEA′, it can be seen that, conceptually, the shaded vibration element PE′x and the white vibration element PE′y are alternately arranged.

In the vibration element array PEA′, in a case of viewing the transmission direction of the grating beam, the phases of the grating beam formed by the ultrasound wave output from the vibration element PE′x and the grating beam formed by the ultrasound wave output from the vibration element PE′y adjacent to the vibration element PE′x are inverted with each other, and thus both grating beams are canceled. Accordingly, by setting the vibration element pitch P to half of p0 satisfying the generation condition of grating beam, the intensity of the grating beam can be reduced.

In the embodiment disclosed in the present specification, the intensity of the grating beam is reduced by using the above-described cancellation condition of the grating beam.

### First Embodiment

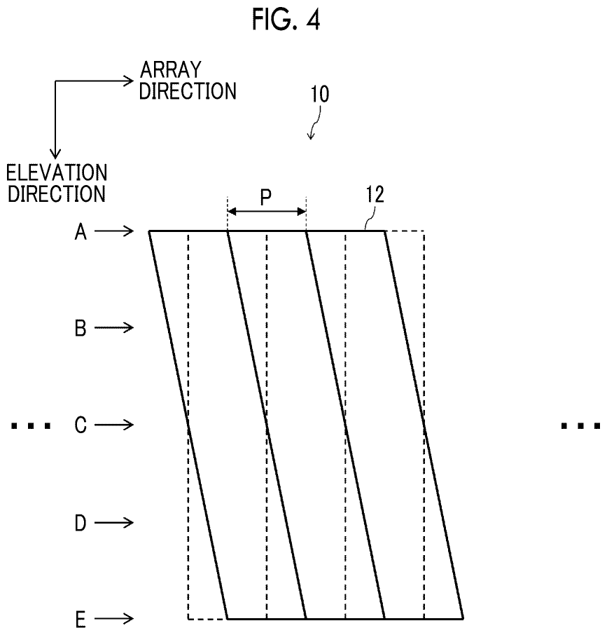

FIG. 3 is a side view of the vibration element array 10 of the ultrasound probe according to the first embodiment, and FIG. 4 is a plan view of the vibration element array 10 of the ultrasound probe according to the first embodiment. The ultrasound probe is used by being connected to an ultrasound diagnostic apparatus main body. The ultrasound probe has a vibration element array 10 including a plurality of vibration elements 12. In a case where a transmission signal, which is an electric signal, is supplied from the ultrasound diagnostic apparatus main body to each vibration element 12, an ultrasound waves having a transmission frequency (or wavelength) corresponding to the transmission signal are output from each vibration element 12. In addition, each vibration element 12 receives reflected waves of the output ultrasound waves from the subject and outputs a reception signal, which is an electric signal, to the ultrasound diagnostic apparatus main body. In the ultrasound diagnostic apparatus main body, various types of processing such as the formation of an ultrasound image (ultrasound tomographic image or Doppler image) and various measurements are executed based on the reception signal.

As shown in FIG. 3, the vibration element array 10 according to the first embodiment is configured with a plurality of vibration elements 12 arranged on a plane. In the present specification, the vibration element array configured with the plurality of vibration elements 12 arranged on the plane in this way is referred to as a linear array. That is, the vibration element array 10 according to the first embodiment is a linear array. In addition, as shown in FIGS. 3 and 4, a vibration element pitch in the vibration element array 10 is represented by P.

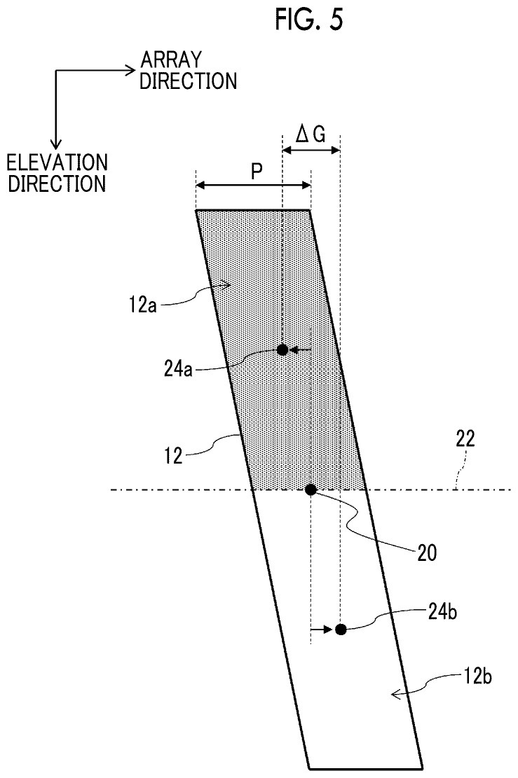

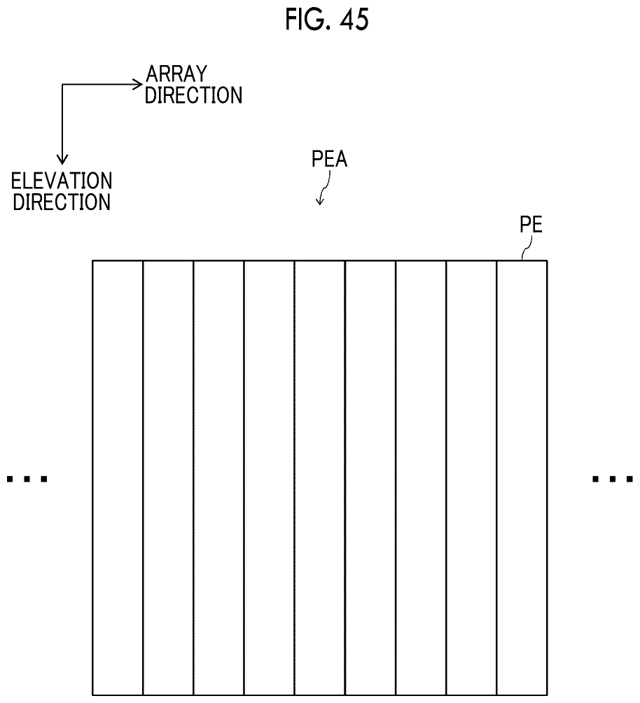

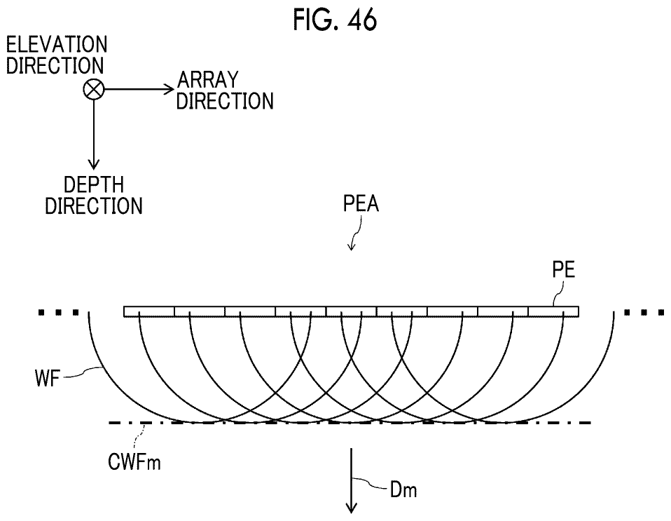

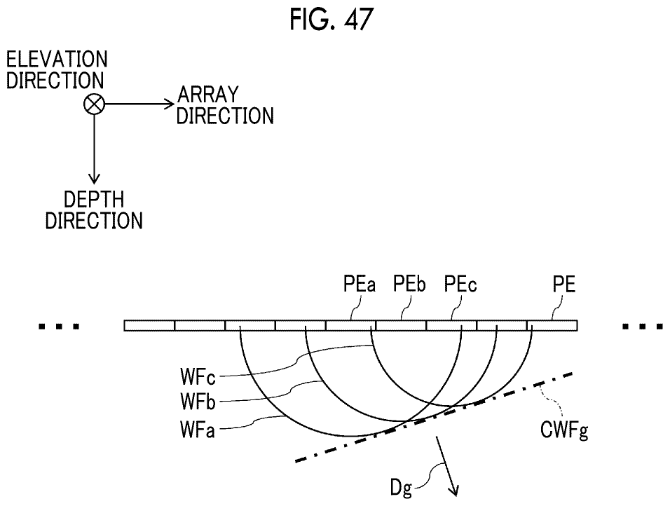

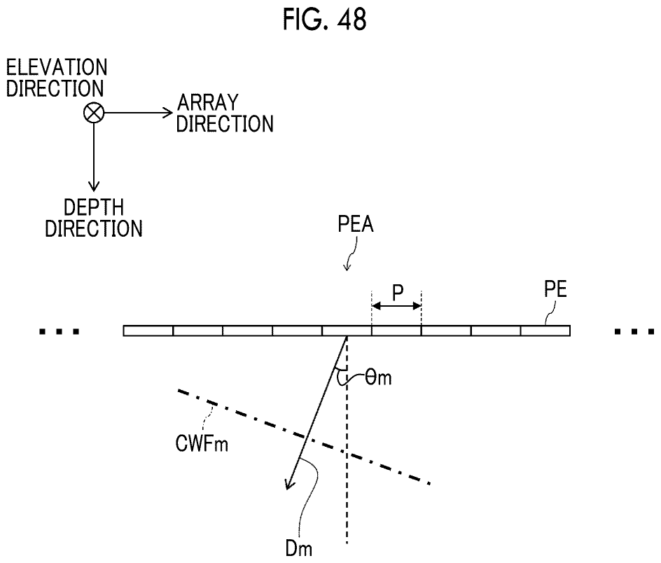

A broken line in FIG. 4 represents, for example, a vibration element in the related art in which the stretching direction is parallel to the elevation direction, as shown in FIG. 45. As shown in FIG. 4, each vibration element 12 included in the vibration element array 10 according to the first embodiment has a shape different from the shape of the vibration element in the related art indicated by a broken line. Hereinafter, details of the shape of the vibration element 12 according to the first embodiment will be described with reference to FIG. 5.

FIG. 5 is an enlarged plan view of the vibration element 12. Here, the vibration element 12 is virtually (conceptually) divided into two portions by a straight line 22 that passes through the centroid 20 of the vibration element 12 and extends in the array direction. In the present specification, in FIG. 5, a portion above the straight line 22 (shaded portion) is referred to as a first virtual portion 12a, and a portion below the straight line 22 (white portion) is referred to as a second virtual portion 12b. In FIG. 5, a first virtual centroid 24a that is a centroid of the first virtual portion 12a and a second virtual centroid 24b that is a centroid of the second virtual portion 12b are shown.

The vibration element 12 has a shape in which the first virtual centroid 24a and the second virtual centroid 24b are at different positions in the array direction. In FIG. 5, the first virtual centroid 24a is shifted to the left side in the array direction with respect to the centroid 20, and the second virtual centroid 24b is shifted to the right side in the array direction with respect to the centroid 20. As a result, the first virtual centroid 24a and the second virtual centroid 24b are shifted by ΔG in the array direction. In the present specification, a distance between the first virtual centroid 24a and the second virtual centroid 24b in the array direction is referred to as an intervirtual centroid distance ΔG, and the intervirtual centroid distance ΔG is expressed in units of the vibration element pitch P (for example, 0.5P).

As an example of a shape in which the first virtual centroid 24a and the second virtual centroid 24b are at different positions in the array direction, in the plan view as seen from the depth direction, the vibration element 12 may have a shape in which the shape of the first virtual portion 12a and the shape of the second virtual portion 12b are point-symmetric with respect to the centroid 20 of the vibration element 12. In the example of FIG. 5, the vibration element 12 has a quadrangular shape (particularly, a parallelogram). In a case where the vibration element 12 is a parallelogram, the first virtual centroid 24a and the second virtual centroid 24b are at different positions in the array direction, so that the stretching direction of the vibration element 12 is not parallel to the elevation direction, and the vibration element 12 has a shape that extends obliquely with respect to the elevation direction from top to bottom in FIG. 5.

Hereinafter, the effect of the vibration element 12 having the above-described shape will be described. The ultrasound waves transmitting and receiving surface of the vibration element 12 has a certain area, and actually, ultrasound waves are output from the entire ultrasound waves transmitting and receiving surface. However, here, it is simply considered that ultrasound waves are output from the first virtual centroid 24a for the first virtual portion 12a and from the second virtual centroid 24b for the second virtual portion 12b. Then, since the first virtual centroid 24a and the second virtual centroid 24b are present at different positions (shifted) from each other in the array direction, as explained with reference to FIGS. 1 and 2, the phases of the grating beam formed by the ultrasound wave output from the first virtual centroid 24a and the grating beam formed by the ultrasound wave output from the second virtual centroid 24b shift from each other, and the two grating beams cancel each other out due to the phase shift. As a result, the intensity of the grating beam is reduced. In particular, in a case where the intervirtual centroid distance ΔG is set to P/2, that is, half of the vibration element pitch, the phases of the grating beam formed by the ultrasound waves output from the first virtual centroid 24a and the grating beam formed by the ultrasound waves output from the second virtual centroid 24b are inverted with each other. Therefore, both the grating beams are canceled, and the intensity of the grating beam is most reduced. That is, the optimum value of the intervirtual centroid distance ΔG is P/2.

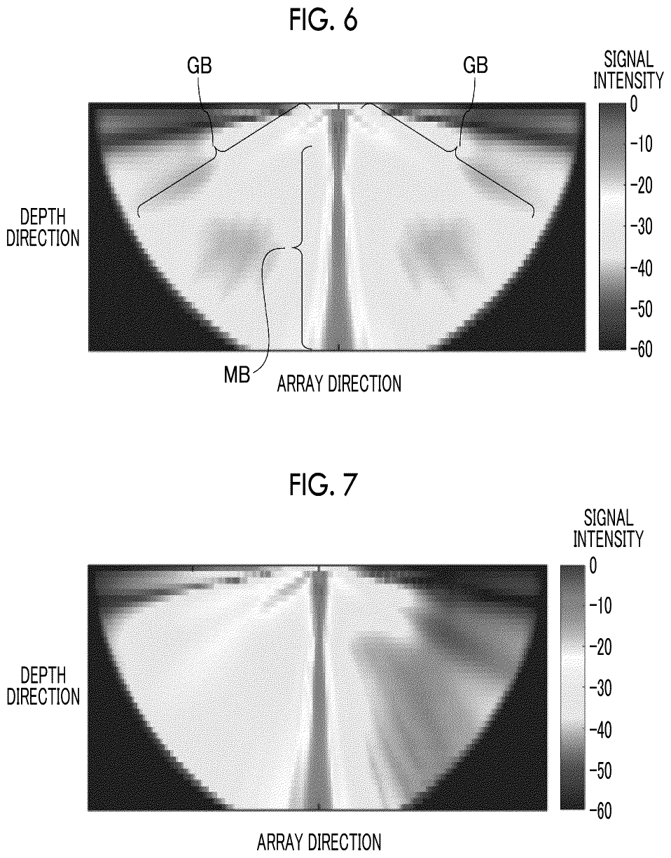

Hereinafter, the verification results of the intensity of the grating beam in a case where the intervirtual centroid distance ΔG is set to P/2 will be described with reference to FIGS. 6 to 20. In the following verification, the intensity of the grating beam in a case where the direction of the main beam is set to a direction parallel to the depth direction is verified.

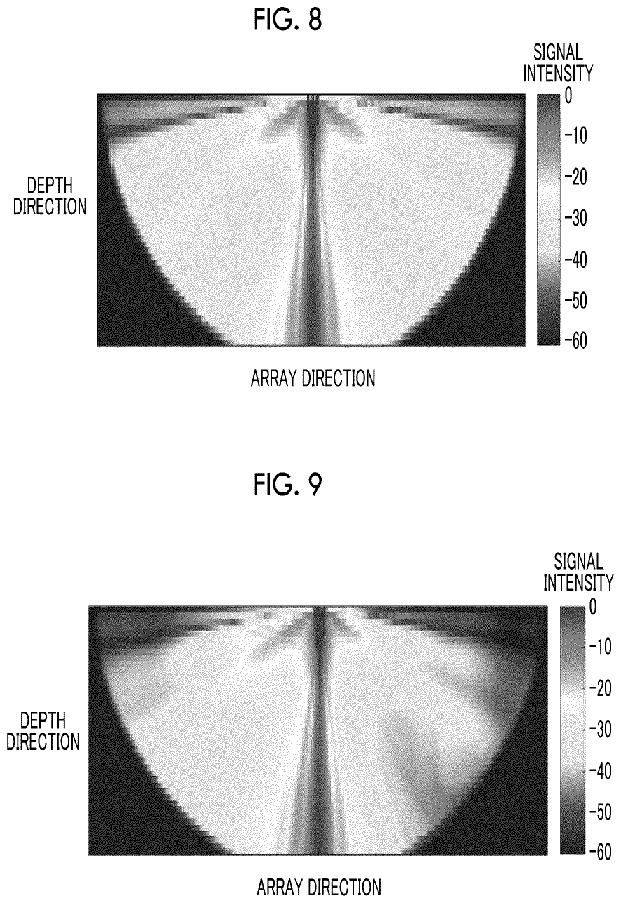

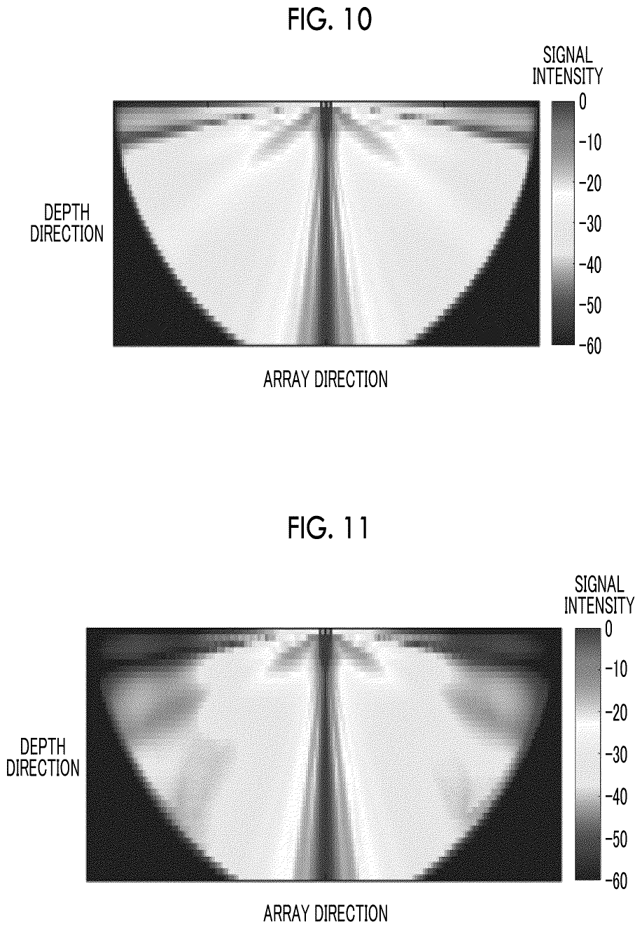

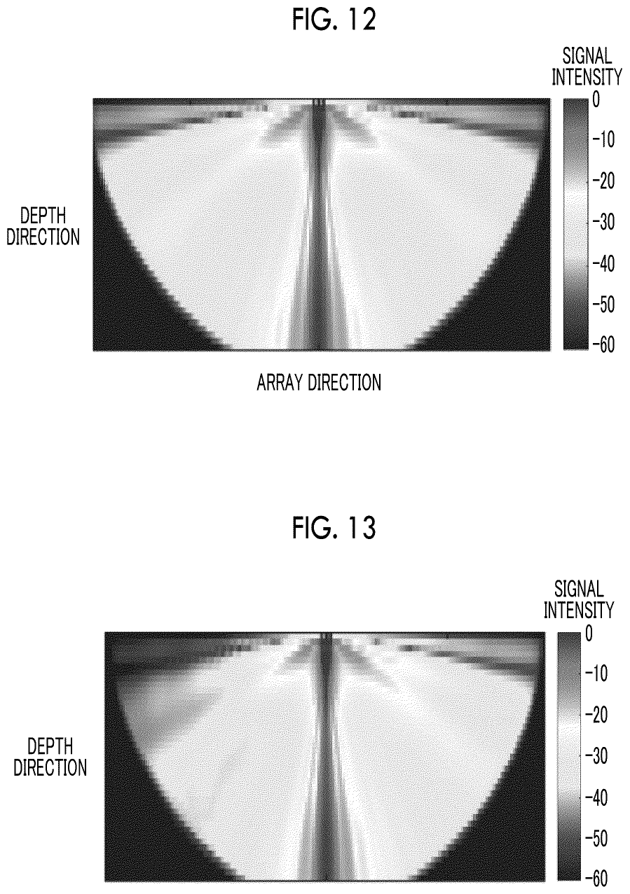

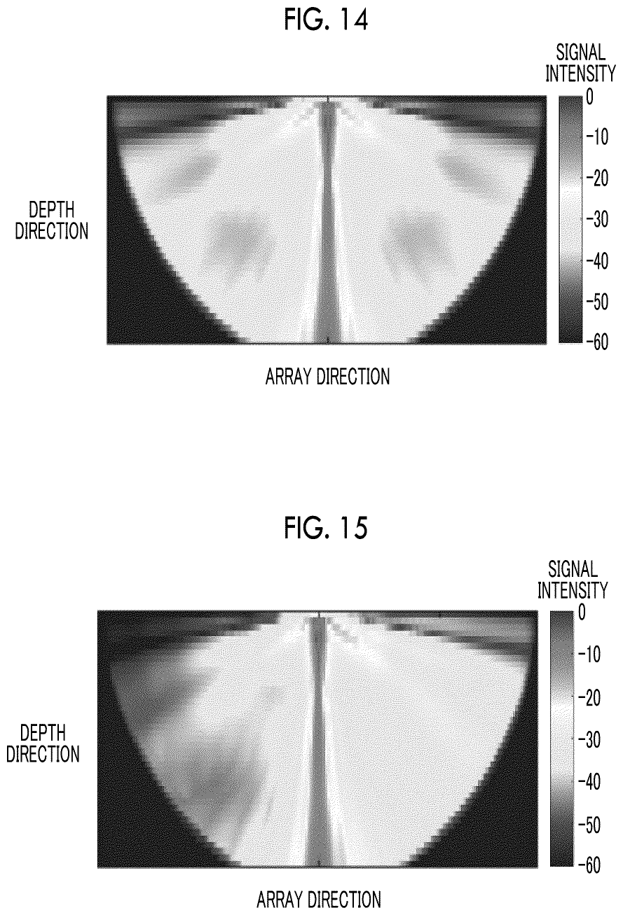

FIG. 6 is a diagram showing a grating beam generated in a linear array in the related art (indicated by a broken line in FIG. 4) at a position in an elevation direction (one end of the vibration element 12 in the elevation direction) indicated by A in FIG. 4. FIG. 7 is a diagram showing a grating beam generated in the vibration element array 10 at a position in the elevation direction indicated by A in FIG. 4. FIG. 8 is a diagram showing a grating beam generated in a linear array in the related art at a position in the elevation direction indicated by B in FIG. 4. FIG. 9 is a diagram showing a grating beam generated in the vibration element array 10 at a position in the elevation direction indicated by B in FIG. 4. FIG. 10 is a diagram showing a grating beam generated in a linear array in the related art at a position in the elevation direction (the center of the vibration element 12 in the elevation direction) indicated by C in FIG. 4. FIG. 11 is a diagram showing a grating beam generated in the vibration element array 10 at a position in the elevation direction indicated by C in FIG. 4. FIG. 12 is a diagram showing a grating beam generated in a linear array in the related art at a position in the elevation direction indicated by D in FIG. 4. FIG. 13 is a diagram showing a grating beam generated in the vibration element array 10 at a position in the elevation direction indicated by D in FIG. 4. FIG. 14 is a diagram showing a grating beam generated in a linear array in the related art at a position in the elevation direction (the other end of the vibration element 12 in the elevation direction) indicated by E in FIG. 4. FIG. 15 is a diagram showing a grating beam generated in the vibration element array 10 at a position in the elevation direction indicated by E in FIG. 4.

In FIG. 6, a portion that extends in the depth direction at the center in the array direction and has a high signal intensity represents the main beam MB, and a portion that extends radially from a depth of 0 at the center in the array direction at a predetermined beam angle and has a slightly high signal intensity represents the grating beam GB. Although the beam directions of the main beam MB and the grating beam GB are typically shown in FIG. 6, the directions of the main beam and the grating beam are also the same in FIGS. 7 to 15 and FIGS. 18 and 19.

By comparing FIG. 6 and FIG. 7, FIG. 8 and FIG. 9, FIG. 10 and FIG. 11, FIG. 12 and FIG. 13, and FIG. 14 and FIG. 15, it can be seen that the intensity of the grating beam GB is reduced by the vibration element array 10 according to the first embodiment. In the center (FIGS. 10 and 11) in the elevation direction, the intensity of the grating beam is particularly reduced. On the other hand, at the end part (FIGS. 6 and 7, and FIGS. 14 and 15) in the elevation direction, the amount of reduction of the grating beam is small on one side of the main beam (the left side of the main beam in FIG. 7 and the right side of the main beam in FIG. 15).

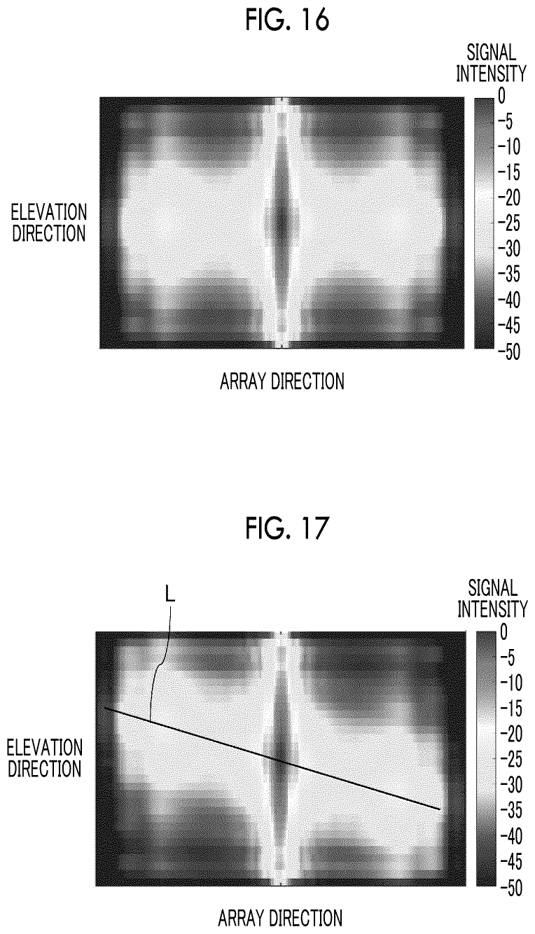

FIG. 16 is a diagram showing a grating beam at a predetermined depth for a linear array in the related art in a two-dimensional space of an array direction and an elevation direction. FIG. 17 is a diagram showing a grating beam at a predetermined depth for the vibration element array 10 in a two-dimensional space of the array direction and the elevation direction. In the vibration element array 10, since the stretching direction of each vibration element 12 extends obliquely with respect to the elevation direction, the generation direction of the grating beam is also inclined as shown by the straight line L in FIG. 17.

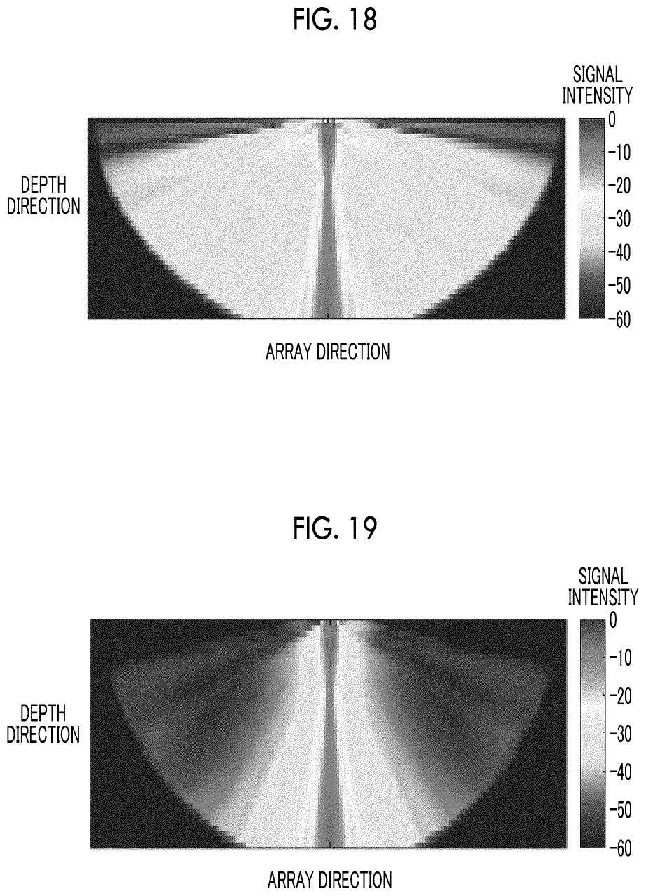

FIG. 18 is a diagram showing a grating beam generated in a linear array in the related art as a whole in the elevation direction. FIG. 19 is a diagram showing a grating beam generated in the vibration element array 10 as a whole in the elevation direction. In FIGS. 6 to 15, the grating beams at each position in the elevation direction are compared, but FIGS. 18 and 19 are diagrams obtained by averaging the signal intensities at the positions in each elevation direction. By comparing FIGS. 18 and 19, it can be seen that the grating beam is significantly reduced as a whole in the elevation direction, that is, as a whole of the vibration element array 10.

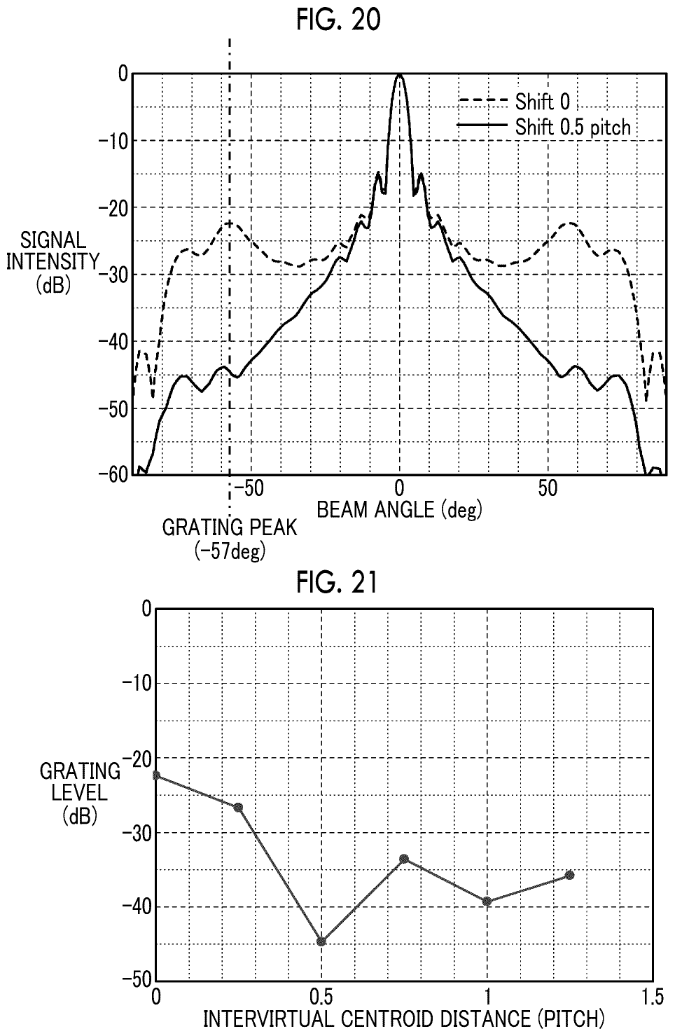

FIG. 20 is a graph showing a relationship between a signal intensity and a beam angle at a predetermined depth. In FIG. 20, a graph indicated by a broken line is a graph for a vibration element in the related art in which the stretching direction is parallel to the elevation direction, and a graph indicated by a solid line is a graph for the vibration element array 10 according to the first embodiment. In the graph of the broken line, the intensity of the grating beam is significantly increased with the beam angle of ±57 degrees as the grating peak, but it can be seen that the grating beam is significantly reduced in the graph of the solid line.

FIG. 21 is a graph showing a relationship between a grating level and an intervirtual centroid distance ΔG at a beam angle at which the intensity of the grating beam is a peak. In the graph of FIG. 21, the case where the intervirtual centroid distance ΔG is 0 (left end of the graph) shows the same shape as the vibration element in the related art in which the stretching direction is parallel to the elevation direction. It can be seen that, at least, the grating level is reduced in a range of the intervirtual centroid distance ΔG shown in FIG. 21 as compared with a case where the intervirtual centroid distance ΔG is 0 (related art). In addition, as described above, the grating level is most reduced in a case where the intervirtual centroid distance ΔG is P/2.

As described above, by setting the shape of the vibration element 12 such that the first virtual centroid 24a and the second virtual centroid 24b are positioned at different positions in the array direction, the grating level can be reduced regardless of the intervirtual centroid distance ΔG. However, from a viewpoint other than the viewpoint of reducing the intensity of the grating level (particularly, from a viewpoint of deterioration in performance of the main beam), an appropriate range (appropriate upper limit) of the intervirtual centroid distance ΔG is present.

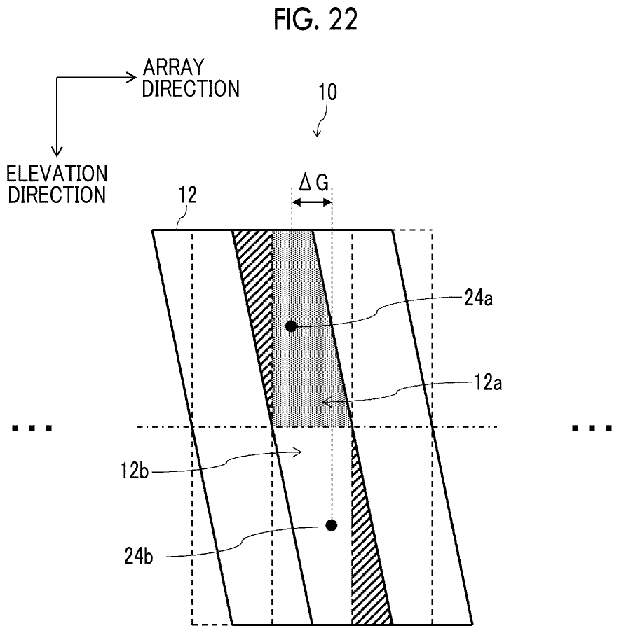

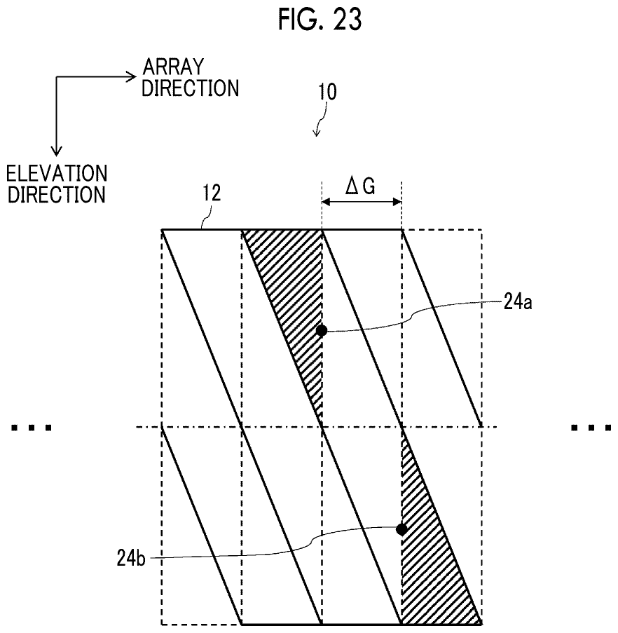

FIGS. 22 and 23 are diagrams for explaining an appropriate upper limit of the intervirtual centroid distance ΔG. As described above, the output timing of the ultrasound waves from each vibration element 12 is controlled in order to control the direction of the main beam. That is, different delay amounts are set for each vibration element 12, and ultrasound waves are output from each vibration element 12 at timings corresponding to the delay amounts. In order to suitably control the direction of the main beam (in other words, in order to output the main beam having a strong signal intensity at a desired beam angle), it is important that the ultrasound waves are output from each position (each sound source) in the array direction with an appropriate delay amount. As shown by a broken line in FIGS. 22 and 23, in the vibration element of the related art in which the stretching direction is parallel to the elevation direction, since the stretching direction is parallel to the elevation direction, the vibration elements 12 as sound sources are aligned in the array direction. Therefore, by setting different delay amounts for each vibration element 12, the direction of the main beam can be suitably controlled.

In a case where the first virtual centroid 24a and the second virtual centroid 24b are at different positions in the array direction, the first virtual portion 12a and the second virtual portion 12b are actually one vibration element 12. Therefore, the first virtual centroid 24a and the second virtual centroid 24b are at different positions in the array direction, but the ultrasound waves are output with the same delay amount (at the same timing). In practice, the ultrasound waves output from the region indicated by the oblique line in FIG. 22 is an ultrasound wave output at a timing different from the target delay amount. This is a factor that deteriorates the accuracy of the direction control of the main beam.

As shown in FIG. 23, as the intervirtual centroid distance ΔG increases (in the example shown in FIG. 23, the intervirtual centroid distance ΔG is one pitch), the hatched region, that is, the region in which the ultrasound wave is output at a timing different from the target delay amount is increased. In other words, as the intervirtual centroid distance ΔG increases, the accuracy of the direction control of the main beam deteriorates. Therefore, the direction control of the main beam and the reduction of the grating level are in a trade-off relationship. Thus, it is preferable to determine the intervirtual centroid distance ΔG in a range in which the balance between both is appropriately maintained. In general, in a case where the intervirtual centroid distance ΔG is equal to or greater than one pitch, half or more of the vibration elements 12 are in the hatched region, and the performance of the main beam deteriorates. Therefore, the intervirtual centroid distance ΔG may be less than the vibration element pitch in the vibration element array 10.

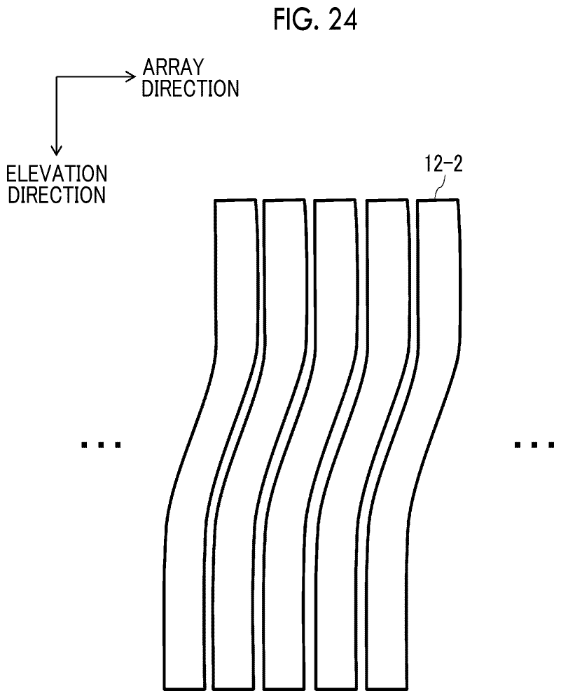

In the examples of FIGS. 4 and 5, the vibration element 12 has a parallelogram shape, but the shape of the vibration element 12 may be other shapes as long as the first virtual centroid 24a and the second virtual centroid 24b are at different positions in the array direction. For example, in the plan view as seen from the depth direction, the shape of the vibration element 12 may be a polygon with 5 or more sides. Alternatively, as in the vibration element 12-2 shown in FIG. 24, at least one of the sides of the vibration element 12-2 in plan view may be curved. Further, in the examples of FIGS. 4 and 5, in a plan view as seen from the depth direction, the vibration element 12 has a shape in which the first virtual portion 12a and the second virtual portion 12b are arranged in a point-symmetric relationship with respect to the centroid 20. However, the vibration element 12 may also have, in a plan view as seen from the depth direction, a shape in which the first virtual portion 12a and the second virtual portion 12b are arranged in a point “non”-symmetric relationship with respect to the centroid 20.

### Second Embodiment

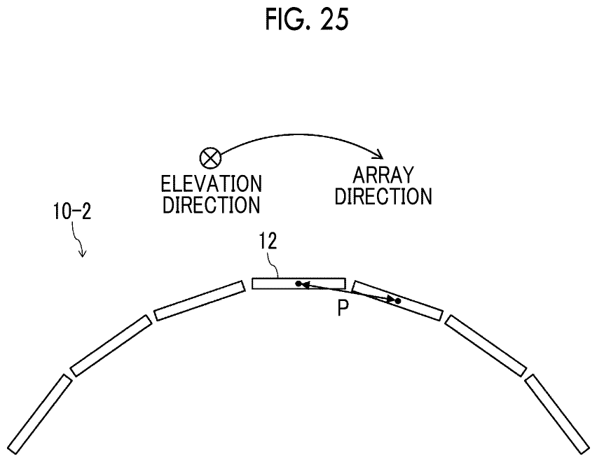

FIG. 25 is a side view of a vibration element array 10-2 of an ultrasound probe according to the second embodiment. As shown in FIG. 25, a vibration element array 10-2 according to the second embodiment is configured with a plurality of vibration elements 12 arranged on a curved surface. In the present specification, the vibration element array configured with the plurality of vibration elements 12 arranged on the curved surface in this way is referred to as a curved surface array. That is, the vibration element array 10-2 according to the second embodiment is a curved surface array. In addition, as shown in FIG. 25, the vibration element pitch in the vibration element array 10-2 is also represented by P. In the curved surface array, the vibration element pitch is represented by a distance in a radial direction between the centroids of the adjacent vibration elements 12-2. In the curved surface array, the array direction is curved. Therefore, in the present specification, a direction perpendicular to the depth direction is referred to as a lateral direction. In the curved surface array, the normal lines of the ultrasound wave transmitting and receiving surfaces of each vibration element 12 are directed in different directions. Therefore, the depth direction and the lateral direction are different for each vibration element.

Even in the second embodiment, similarly to the first embodiment, each of the vibration elements 12 has a shape in which the first virtual centroid 24a and the second virtual centroid 24b are at different positions in the array direction, for example, as shown in FIGS. 4 and 5 in the plan view as seen from the depth direction. As a result, the intensity of the grating beam output from the vibration element array 10-2 is reduced by the same principle as that of the first embodiment.

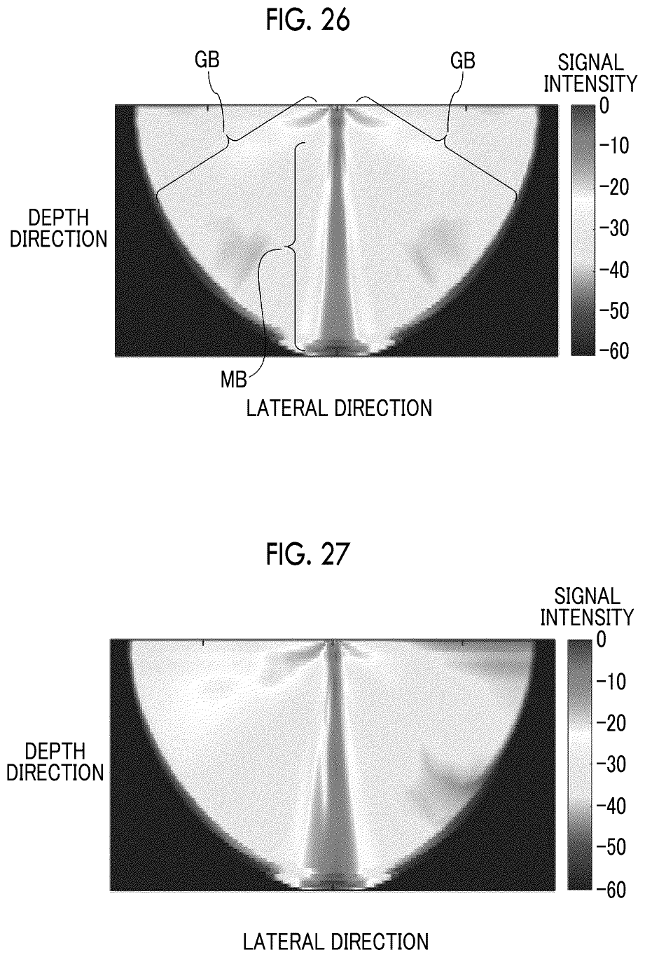

Hereinafter, the verification results of the intensity of the grating beam in a case where the intervirtual centroid distance ΔG is set to P/2 will be described with reference to FIGS. 26 to 40. In the following verification, the intensity of the grating beam in a case where the direction of the main beam is set to a direction parallel to the depth direction of one vibration element 12 is verified.

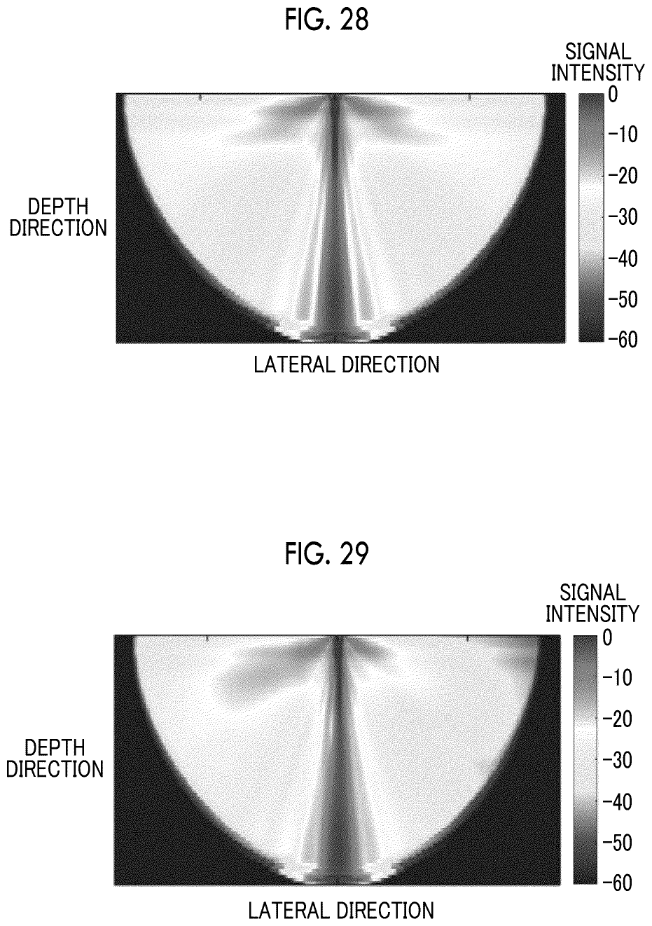

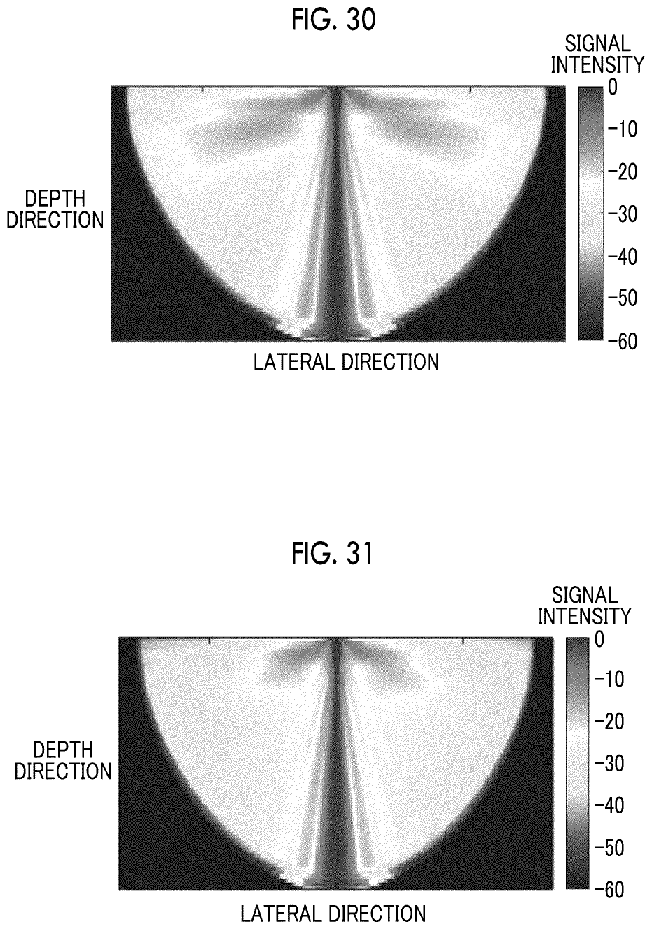

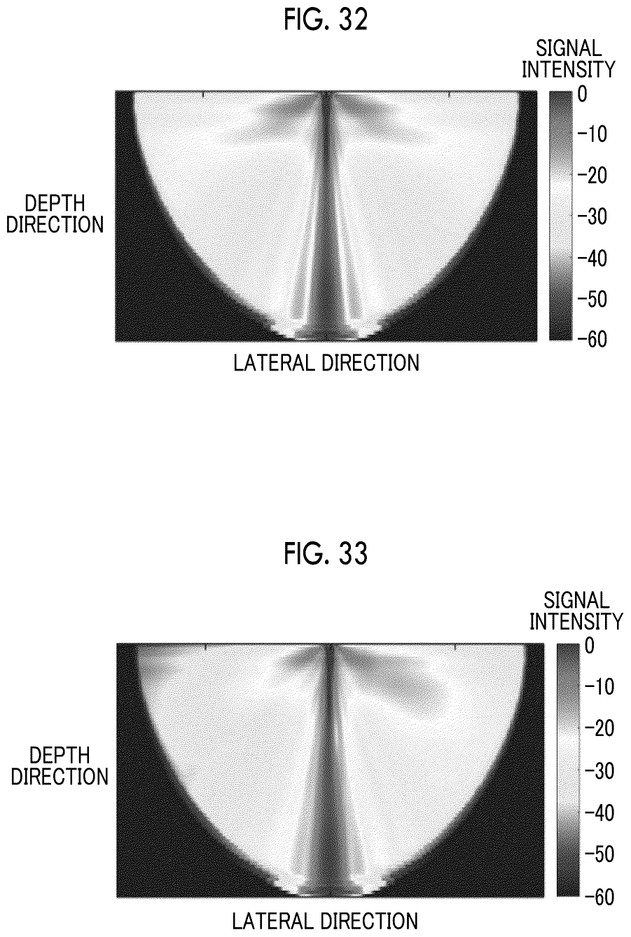

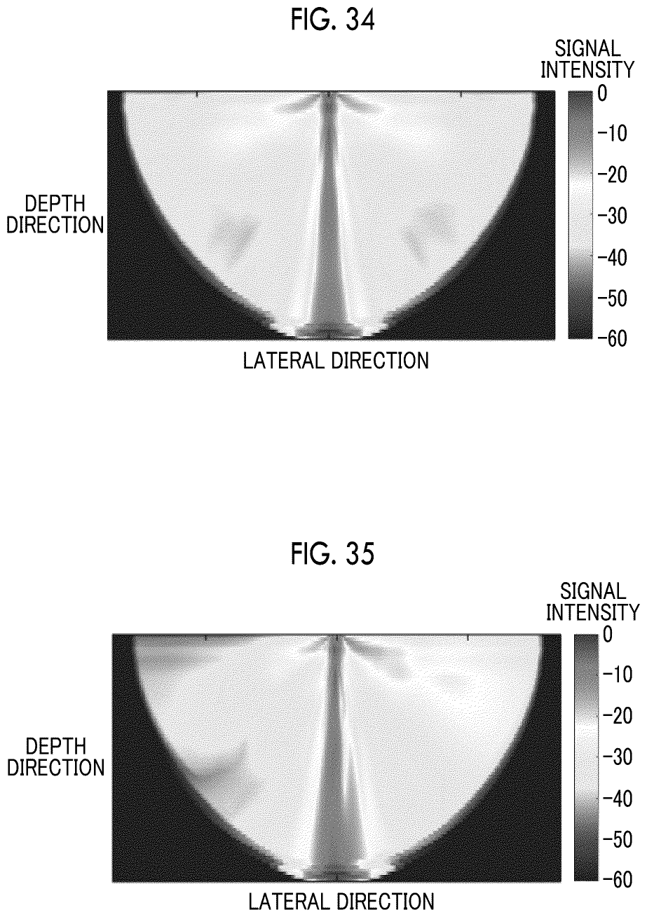

FIG. 26 is a diagram showing a grating beam generated in a curved surface array (indicated by a broken line in FIG. 4) in the related art at a position in the elevation direction (one end of the vibration element 12 in the elevation direction) indicated by A in FIG. 4. FIG. 27 is a diagram showing a grating beam generated in the vibration element array 10-2 at a position in the elevation direction indicated by A of FIG. 4. FIG. 28 is a diagram showing a grating beam generated in the curved surface array in the related art at a position in the elevation direction indicated by B in FIG. 4. FIG. 29 is a diagram showing a grating beam generated in the vibration element array 10-2 at a position in the elevation direction indicated by B in FIG. 4. FIG. 30 is a diagram showing a grating beam generated in the curved surface array in the related art at a position in the elevation direction (the center of the vibration element 12 in the elevation direction) indicated by C in FIG. 4. FIG. 31 is a diagram showing a grating beam generated in the vibration element array 10-2 at a position in the elevation direction indicated by C in FIG. 4. FIG. 32 is a diagram showing a grating beam generated in the curved surface array in the related art at a position in the elevation direction indicated by D in FIG. 4. FIG. 33 is a diagram showing a grating beam generated in the vibration element array 10-2 at a position in the elevation direction indicated by D in FIG. 4. FIG. 34 is a diagram showing a grating beam generated in the curved surface array in the related art at a position in the elevation direction (the other end of the vibration element 12 in the elevation direction) indicated by E in FIG. 4. FIG. 35 is a diagram showing a grating beam generated in the vibration element array 10-2 at a position in the elevation direction indicated by E in FIG. 4.

In FIG. 26, a portion that extends in the depth direction at the center in the array direction and has a high signal intensity represents the main beam MB, and a portion that extends radially from the depth 0 at the center in the array direction at a predetermined beam angle and has a slightly high signal intensity represents the grating beam GB. Although the beam directions of the main beam MB and the grating beam GB are typically shown in FIG. 26, the directions of the main beam and the grating beam are also the same in FIGS. 27 to 35 and FIGS. 38 and 39.

By comparing FIG. 26 and FIG. 27, FIG. 28 and FIG. 29, FIG. 30 and FIG. 31, FIG. 32 and FIG. 33, and FIG. 34 and FIG. 35, it can be seen that the intensity of the grating beam GB is also reduced by the vibration element array 10-2 according to the second embodiment. As in the first embodiment, even in the second embodiment, the intensity of the grating beam is particularly reduced at the center (FIGS. 30 and 31) in the elevation direction. On the other hand, at the end part in the elevation direction (FIGS. 26 and 27, and FIGS. 34 and 35), the amount of reduction of the grating beam is small on one side of the main beam (the left side of the main beam in FIG. 27 and the right side of the main beam in FIG. 35).

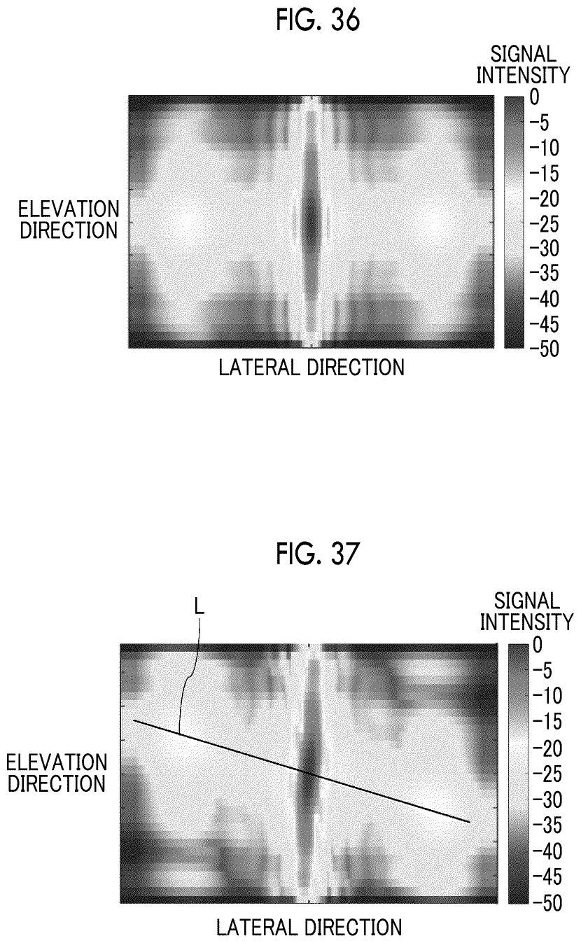

FIG. 36 is a diagram showing a grating beam at a predetermined depth of a curved surface array in the related art in a two-dimensional space of a lateral direction and an elevation direction. FIG. 37 is a diagram showing a grating beam at a predetermined depth for the vibration element array 10-2 in a two-dimensional space of the lateral direction and the elevation direction. As in the first embodiment, in the vibration element array 10-2, since the stretching direction of each vibration element 12 extends obliquely with respect to the elevation direction, the generation direction of the grating beam is also inclined as shown by the straight line L in FIG. 37.

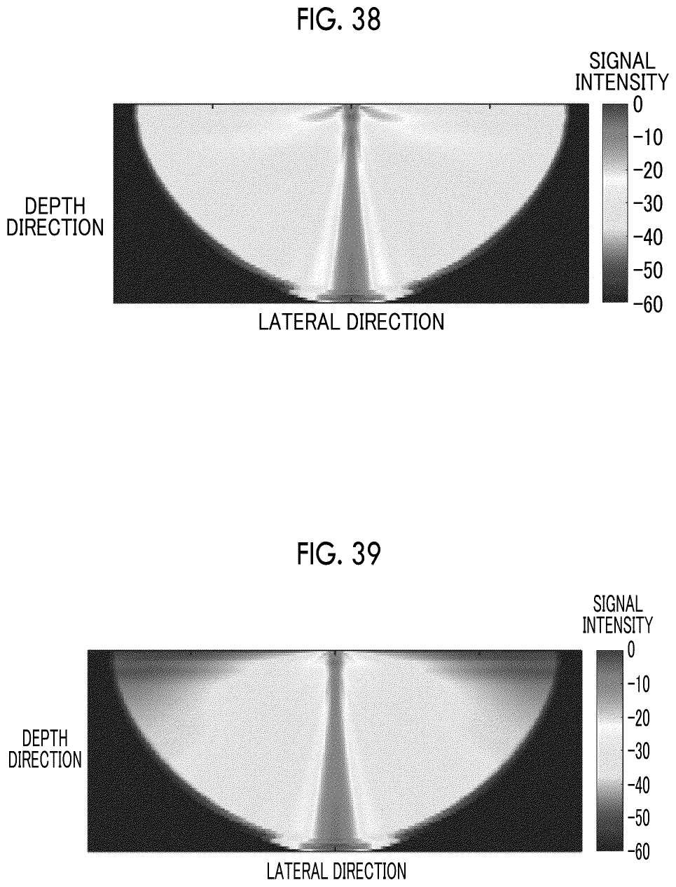

FIG. 38 is a diagram showing a grating beam generated in a curved surface array in the related art as a whole in the elevation direction. FIG. 39 is a diagram showing a grating beam generated in the vibration element array 10-2 as a whole in the elevation direction. FIGS. 26 to 35 show a comparison of the grating beams at each position in the elevation direction, but FIGS. 38 and 39 are diagrams obtained by averaging the signal intensities at the positions in each elevation direction. It can be seen that the grating beam is significantly reduced as a whole in the elevation direction, that is, as a whole of the vibration element array 10-2, by comparing FIGS. 38 and 39.

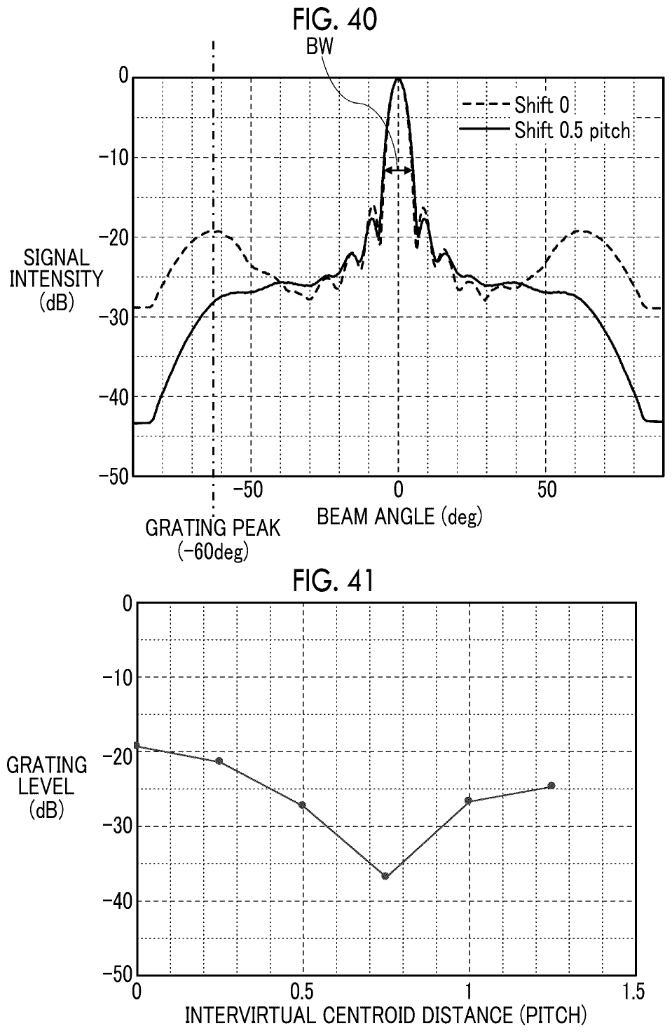

FIG. 40 is a graph showing a relationship between a signal intensity and a beam angle at a predetermined depth. In FIG. 40, a graph indicated by a broken line is a graph for a vibration element in the related art in which the stretching direction is parallel to the elevation direction, and a graph indicated by a solid line is a graph for the vibration element array 10-2 according to the second embodiment. In the graph of the broken line, the intensity of the grating beam is significantly increased with the beam angle of about ±60 degrees as the grating peak, but it can be seen that the grating beam is significantly reduced in the graph of the solid line.

FIG. 41 is a graph showing a relationship between a grating level and an intervirtual centroid distance ΔG at a beam angle at which the intensity of the grating beam is a peak. In the graph of FIG. 41, the case where the intervirtual centroid distance ΔG is 0 (the left end of the graph) shows the same shape as the vibration element in the related art in which the stretching direction is parallel to the elevation direction. It can be seen that, at least, the grating level is reduced within the range of the intervirtual centroid distance ΔG shown in FIG. 41 as compared with a case where the intervirtual centroid distance ΔG is 0 (related art).

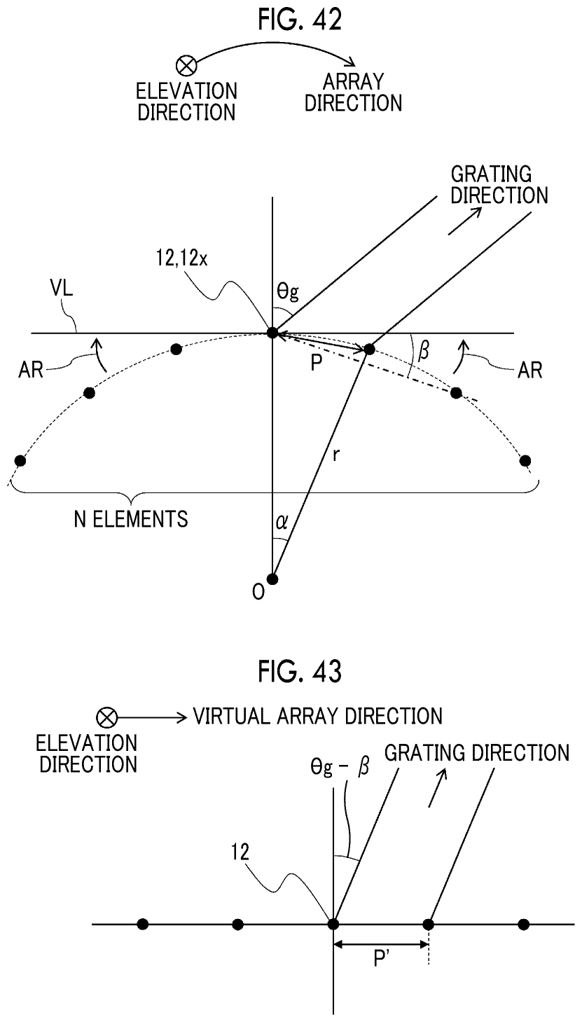

In the first embodiment (linear array), the grating level is most reduced in a case where the intervirtual centroid distance ΔG is P/2. However, in the second embodiment (curved surface array), as shown in FIG. 41, the grating level is most reduced in a case where the intervirtual centroid distance ΔG is not P/2 (0.5 pitches) but about 0.75 pitches. Hereinafter, a method of obtaining an optimal intervirtual centroid distance ΔG in the vibration element array 10-2, which is the curved surface array, will be described with reference to FIGS. 42 and 43 from the viewpoint of reducing the grating level.

FIGS. 42 and 43 are diagrams for explaining a method of calculating a virtual pitch. In FIG. 42, each vibration element 12 is simply shown by a black circle indicating the position of the centroid thereof. In the following description, as shown in FIG. 42, a curvature radius of a curved surface on which the vibration elements 12 are arranged is r (a center thereof is a point O), an angular pitch between the vibration elements 12 is α, the number of diameter vibration elements used during transmission or reception of the vibration element array 10-2 is N, and a beam angle of a grating beam with respect to a direction of a main beam is θg in a case where a direction perpendicular to a virtual straight line VL (directly above in FIG. 42) from the vibration element 12x is defined as the direction of the main beam.

The vibration element array 10-2 is a curved surface array, and the vibration elements 12 are arranged on the curved surface. Therefore, it is difficult to obtain the optimal intervirtual centroid distance ΔG as it is. Therefore, in the present embodiment, as shown in FIG. 43, each of the vibration elements 12 arranged on the curved surface is converted into a virtual linear array (virtual linear array), and the optimal intervirtual centroid distance ΔG is obtained by using the vibration element pitch in the virtual linear array (referred to as a virtual pitch in the present specification).

Specifically, a virtual straight line VL that is a tangent line of a curved surface on which each vibration element 12 is arranged and that passes through one vibration element 12 (in FIG. 42, the vibration element 12x) is assumed. Then, the vibration element 12 other than the vibration element 12x is virtually rotated as indicated by the arrow AR and is virtually moved on the virtual straight line VL to form the virtual linear array. Here, the rotation angle of each vibration element 12 can be approximately the average angle of the rotation angles of the vibration elements 12 having half of the aperture of the vibration element array 10-2. In a case where the average angle is denoted by β, β can be represented by the following Expression 2.

$$β = ( N / 4 ) * α \qquad Expression 2$$

That is, by rotating each of the vibration elements 12 by the angle β, it is possible to form a virtual linear array in which the vibration elements 12 are virtually arranged on the virtual straight line VL.

On the other hand, the generation condition of the grating beam in the linear array can be represented by the following Expression 3.

$$P \cdot sin θ = m λ \qquad Expression 3$$

In Expression 3, P is a vibration element pitch, θ is a beam angle of a grating beam, m is an integer, and λ is a wavelength of the ultrasound wave.

In a case where Expression 3 is modified, Expression 4 is obtained.

$$P = m λ / sin θ \qquad Expression 4$$

Expression 4 shows that in a case where the beam angle θ of the grating beam is small, the vibration element pitch P increases under the generation condition of the grating beam. As shown in Expression 2, it can be approximated that the grating angle is θg−β as the virtual linear array is formed by virtually rotating each of the vibration elements 12 by the angle β (refer to FIG. 43). Therefore, assuming that ma is constant from Expression 3,

$$P ’ sin ( θ - β ) = P sin θ g \qquad Expression 5$$

is established.

In Expression 5, P′ is the vibration element pitch in the virtual linear array, that is, the virtual pitch.

From Expression 5, the virtual pitch P′ is represented by Expression 6.

$$P ’ = P sin θ g / sin ( θ g - β ) \qquad Expression 6$$

Since the virtual pitch P′ in the virtual linear array is obtained by Expression 6, the intensity of the grating beam can be most reduced by setting the intervirtual centroid distance ΔG to half of the virtual pitch P′ from the first embodiment.

In Expression 6, in a case where the angle β is sufficiently larger than the angle 0g, Expression 6 can be approximately represented by Expression 7.

$$P ’ = P / ( 1 - β ) \qquad Expression 7$$

As the virtual pitch P′, the optimal intervirtual centroid distance ΔG may be determined using Expression 7 in a simplified manner.

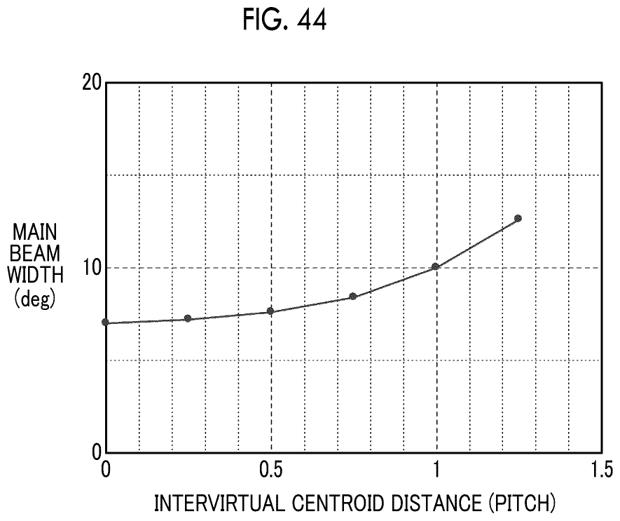

FIG. 44 is a graph showing a relationship between a main beam width and an intervirtual centroid distance ΔG in the vibration element array 10-2. The main beam width means, with a beam angle at which the main beam has the maximum signal intensity as a reference, an angle width of the beam at a signal intensity smaller than the maximum signal intensity of the main beam by a predetermined amount (see reference BW in FIG. 40). It can be said that the smaller the main beam width BW, the better the performance of the main beam.

As shown in FIG. 44, the main beam width BW increases as the intervirtual centroid distance ΔG increases. That is, the performance of the main beam is degraded. That is, since the intervirtual centroid distance ΔG and the main beam width BW are in a trade-off relationship, it is preferable to determine the intervirtual centroid distance ΔG in consideration of the balance between the two. For example, in a case where the intervirtual centroid distance ΔG is set to 0.5 pitches and a case where the intervirtual centroid distance ΔG is set to 0.75 pitches are compared, the intensity of the grating beam is further reduced in a case where the intervirtual centroid distance ΔG is set to 0.75 pitches than in a case where the intervirtual centroid distance ΔG is set to 0.5 pitches (refer to FIG. 41), but the main beam width BW is increased. In this case, the intervirtual centroid distance ΔG may be set to 0.75 pitches to reduce the intensity of the grating beam, or the intervirtual centroid distance ΔG may be set to 0.5 pitches to emphasize the main beam width BW.

Although the examination result display device of the vibration element according to the present disclosure has been described above, the examination result display device of the vibration element according to the present disclosure is not limited to the above-described embodiment, and various changes can be made without departing from the gist of the present disclosure.

What is claimed is:

1. An ultrasound probe comprising:
a vibration element array including a plurality of vibration elements arranged in an array direction,
wherein, in a case where the vibration elements are virtually divided into a first virtual portion and a second virtual portion by a straight line passing through a centroid of the vibration elements and extending in the array direction, each of the vibration elements has a shape in which a first virtual centroid that is a centroid of the first virtual portion and a second virtual centroid that is a centroid of the second virtual portion are located at different positions in the array direction, and
due to the shape, a grating beam output from the first virtual portion and a grating beam output from the second virtual portion are canceled out from each other due to a phase shift, so that an intensity of the grating beam output from each of the vibration elements is reduced.

2. The ultrasound probe according to claim 1,
wherein, in a plan view as seen from a depth direction, the shape of the vibration element is a polygon with 5 or more sides, or at least one side of the vibration element is curved.

3. The ultrasound probe according to claim 1,
wherein, in a plan view as seen from a depth direction, a shape of the first virtual portion and a shape of the second virtual portion are in a point-asymmetric relationship with the centroid of the vibration element as a center.

4. The ultrasound probe according to claim 1,
wherein an intervirtual centroid distance, which is a distance between the first virtual centroid and the second virtual centroid in the array direction, is less than a vibration element pitch in the vibration element array.

5. The ultrasound probe according to claim 4,
wherein, in a case where the plurality of vibration elements are arranged on a plane, the intervirtual centroid distance is half of the vibration element pitch.

6. The ultrasound probe according to claim 4,
wherein, in a case where the plurality of vibration elements are arranged on a curved surface, the intervirtual centroid distance is half of a virtual pitch P′ between the vibration elements, and
the virtual pitch P′ is represented by
$$P ’ = P sin θ g / sin ( θ g - β )$$
$$β = ( N / 4 ) * α ,$$
here, P is the vibration element pitch, θg is a beam angle of the grating beam, N is the number of diameter vibration elements, and α is an angular pitch between the vibration elements.
